(19) United States
(12) Patent Application Publication
(10) Pub. No.: US 2006/0105946 A9
(43) Pub. Date: May 18, 2006

(54) Method for treating the central nervous system by administration of igf structural analogs

(76) Inventor: Douglas N. Ishii, La Porte, CO (US)

Correspondence Address: Janelle D Waack;Howrey Simon Arnold & White, 750 Bering Drive, Houston, TX, 77057-2198, US

(21) Appl. No.: 10/363,574

(22) PCT Filed: Aug. 28, 2001
(86) PCT No.: PCT/US01/26750

§ 371 Date: Oct. 2, 2003

Related U.S. Application Data
(60) Provisional application No. 60/228,633, filed on Aug. 29, 2000.

(51) Int. Cl. A61K 38/30

(52) U.S. Cl. 514/12

(57) ABSTRACT

This invention is directed to a method for treating a disease or disorder of the brain or spinal cord in a mammal, including human, comprising the administration of an effective amount of an insulin-like growth factor (IGF) structural analog at a site outside of the blood-brain, blood-central nervous system, or blood-spinal cord barrier.

This patent application is based on provisional U.S. patent application Ser. No. 60/228,633, filed Aug. 29, 2000.

The United States Government may own certain rights in the present invention pursuant to National Institute of Neurological Disorders and Stroke Grants Nos. 5 RO1 NS24327 and 9 RO1 DK53922, as well as Centers for Disease Control Grant R49 CCR811509.

### SUMMARY OF THE INVENTION

This invention is directed to a method for treating the central nervous system by the nonintracranial and nonintravertebral column administration of one or more IGF structural analogs. More particularly, the invention is directed to a method for treating disorders or diseases of the brain or spinal cord by the nonintracranial or nonintravertebral column administration of one or more IGF structural analogs.

### DESCRIPTION OF DRAWINGS

FIG. 1. Concentration-dependent detection of (A) hIGF-I, (B) Des(1-3)hIGF-I, (C) [Leu²⁴]hIGF-I and (D) [Leu⁶⁰]hIGF-I by ELISA. Samples were assayed in triplicate at each concentration. The coefficient of correlation, r, was determined by linear regression using a computer software program.

FIG. 2. Dose-dependent distribution of immunoreactive hIGF-I in CSF and plasma following subcutaneous injections in adult rats. Plasma and CSF were withdrawn for ELISA 90 min after a single bolus subcutaneous injection of the indicated dose of hIGF-I, and each sample was assayed in triplicate. Group means±SEM are shown (n=3 rats per dose). Part A, CSF hIGF-I. Part B, plasma hIGF-I. The data were plotted using linear regression, r=0.97.

FIG. 3. Effect of simultaneous administration of hIGF-II on hIGF-I uptake into CSF. Rats were injected subcutaneously with 150 μg hIGF-I alone (n=8) or the combination of 150 μg hIGF-I and 400 μg hIGF-II (n=6). Plasma and CSF were withdrawn 90 min later for assay. Values are means±SEM. The group means were compared using a t-test. *P<0.02.

FIG. 4. Comparative distribution in CSF and plasma following administration of Des(1-3)hIGF-I (n=4), hIGF-I (n=3), or vehicle (n=2). Equivalent amounts (200 μg per rat) of Des(1-3)hIGF-I or hIGF-I were injected subcutaneously, and plasma and CSF were withdrawn for assay 90 min later. Group means were compared using Newman-Keuhl's posthoc test. *P<0.002 and 0.003 for Des and hIGF-I, respectively, vs. control in CSF. *P<0.002 for hIGF-I vs. control in plasma.

FIG. 5. Uptake of [Leu²⁴]hIGF-I and [Leu⁶⁰]hIGF-I into CSF. [Leu²⁴]hIGF-I (200 μg per rat; n=3 rats), [Leu⁶⁰]hIGF-I (100 μg per rat; n=4) or hIGF-I (200 μg per rat; n=3) or vehicle (n=9) were injected subcutaneously, and 90 min later plasma and CSF were withdrawn for assay. Part A, CSF; Part B, plasma. Differences between group means were detected using Newman-Keuhl's posthoc test. *P<0.002 for hIGF-I vs. [Leu²⁴]hIGF-I in CSF. *P<0.0004 for [Leu²⁴] and [Leu⁶⁰] vs. control and 0.0007 for hIGF-I vs. control in CSF. In plasma, *P<0.0002 and 0.0005 for [Leu²⁴] and [Leu⁶⁰], respectively, vs. hIGF-I. *P<0.0005 and 0.0002 for [Leu⁶⁰] and hIGF-I, respectively, vs. control.

### DETAILED DESCRIPTION OF THE INVENTION

This invention is directed to a method for treating the central nervous system by the nonintracranial and nonintravertebral column administration of one or more IGF structural analogs. More particularly, the invention is directed to a method for treating disorders or diseases of the brain or spinal cord by the nonintracranial or nonintravertebral column administration of one or more IGF structural analogs. For purposes of this invention, “IGF structural analogs” are defined as molecules having substantial sequence homology to naturally occurring insulin-like growth factors (IGFs), including human and animal (including but not limited to cow, pig, dog, sheep, horse, deer, goat, rat, mouse and chicken) IGF-I and IGF-II. More preferably, the IGF structural analogs have amino acid sequences of IGF molecules that have been modified by deletions, substitutions and/or additions of fewer than 15 amino acids.

In the method according to the invention, the preferred route of administration of the IGF structural analog is from a site outside of the blood-brain-barrier (BBB), blood-central nervous system-barrier (B-CNS-B) and blood-spinal cord-barrier (B-SC-B). Any of the common routes of administration known to the pharmaceutical sciences may be used that can deliver IGF structural analogs into the circulation, including but not limited to percutaneous, intradermal, subcutaneous, intravenous, intramuscular, intraarterial, intraperitoneal, parenteral, buccal, sublingual, rectal, oral, nasal, by inhalation, from a subcutaneous implanted pump or matrix, or from a plasmid construct containing an IGF structural analog gene that is administered at a site outside of the BBB, B-CNS-B and B-SC-B. For example, the nasal cavity and lung are richly vascularized, and IGF structural analogs administered into the nasal cavity or by inhalation may be rapidly taken up by the local microvasculature, resulting in IGF analogs being taken up into cerebrospinal fluid (CSF) across the BBB or B-CNS-B. This invention is not limited to a particular route of administration, other than that the administration is from a site outside of the BBB, B-CNS-B and B-SC-B.

In a preferred embodiment, an IGF structural analog may be administered alone or in combination with other IGF analogs. The IGF analog may also be combined with one or more excipients, coloring agents, salts, solvents, carriers, stabilizers, and other ingredients that may be used in formulations and are known to the pharmaceutical sciences. In a further preferred embodiment, the IGF structural analog is administered in an amount from about 0.01 μg/kg/day up to about 4 mg/kg/day.

In a preferred embodiment, the invention is directed to a method for treating disorders or diseases of the postbirth brain or spinal cord, such as Alzheimer's Disease, Parkinson's Disease, AIDS-related dementia, senile dementia, stroke, trauma, cortical-basal ganglionic syndromes, progressive dementia, familial dementia with spastic paraparesis, progressive supranuclear palsy, multiple sclerosis, hepatic encephalopathy, Pick's Disease, Huntington's Disease, diffuse cerebral sclerosis of Schilder, acute necrotizing hemorrhagic encephalomyelitis, brain tumors and the like. This invention does not include amyotrophic lateral sclerosis.

IGF structural analogs that may be used in the present invention include but are not limited to des(1-3)IGF-I, which is an IGF-I analog lacking the N-terminal tripeptide; [Arg3]IGF-I, which is an IGF-I analog in which Arg is substituted for Glu at position 3; [Leu24]IGF-I, which is an IGF-I analog in which Leu is substituted for Thr at position 24; [Leu60]IGF-I, which is a mutant IGF-I with Leu substituted for Tyr at position 60; Long R3IGF-I, which is a mutant IGF-I with Arg substituted for Glu at position 3 as well as a 13 amino acid extension at the N-terminus; des(1-6)IGF-II, which is an IGF-11 analog lacking the N-terminal hexapeptide; [Gly1]IGF-II, which is an IGF-11 mutant with Gly substituted for Ala at position 1; [Arg6]IGF-II, which is an IGF-II mutant with an Arg substituted for Glu at position 6; and [Leu27]IGF-II, which is an IGF-IL mutant with Leu substituted for Tyr at position 27. These IGF structural analogs are commercially available, for example, from GroPep, Pty. Ltd. (Australia). It is appreciated that in the art it is possible to produce various additional IGF structural analogs.

The IGF structural analogs used in the present invention have biological activity. For example, it is known that des(1-3)IGF-I administered into the eye can enhance the growth of transplanted spinal cord, cerebral cortex and parietal cortex in the eye. It can increase choline acetyltransferase activity in cultured spinal cord and enhance growth of cultured olfactory bulb cells. [Arg3]IGF-I, long R³IGF-I, [Leu24]IGF-I, [Leu60]IGF-I, des(1-6)IGF-II, [Gly1]IGF-II, [Arg6]IGF-II, and [Leu27]IGF-II can bind to type I IGF receptors, type II IGF receptors, or IGF binding proteins and alter protein synthesis in cells. Thus, IGF structural analogs that cross the BBB, B-CNS-B or B-SC-B may be used for the purposes of this invention.

The treatment of the brain and spinal cord is more complicated than the treatment of the peripheral nervous system because the B-CNS-B, B-SC-B and BBB pose an obstacle to the delivery of pharmaceutical agents, particularly proteins and peptides, to the central nervous system. These barriers are widely believed to prevent the uptake and penetration of proteins and peptides, such as IGFs, and these concerns would apply equally well to IGF structural analogs. Applicant has previously shown that IGF-I or IGF-II can cross from the blood into the CSF and normalize brain biochemistry in disease, prevent loss of axons in the spinal cord, and prevent functional damage to the central nervous system. Therefore, based on subsequent research, it is expected that IGF structural analogs can likewise cross from the blood into the cerebral spinal fluid (CSF) and may prevent damage, disease or disorder in the central nervous system.

The following examples show that IGF structural analogs can enter the CSF from the circulation. Consequently, IGF structural analogs may effect changes in or treat the central nervous system. The examples show that there is a carrier that takes IGFs up from the circulation into CSF, and the properties of this carrier differ from known IGF binding proteins and IGF receptors, such as type I IGF receptor or type II IGF receptor. The IGF analogs in the examples that are taken up into CSF includes des(1-3)IGF-I, [Leu24]IGF-I and [Leu60]IGF-I. Furthermore, IGF-II reduces IGF-I uptake into CSF, and this is consistent with competition for uptake by a common IGF carrier. The invention in its broader aspects is not limited to the specific details or representative examples described. Therefore, based on subsequent research, it is expected that IGF structural analogs that are taken up into CSF by this carrier may be used for the purposes of this invention. Those IGF structural analogs that are taken up into CSF may serve as agonists or antagonists. Antagonists may be useful for inhibiting the growth of brain tumors that may be IGF-dependent, for example. Agonists may be useful for treating various brain diseases and disorders such as Parkinson's Disease, Alzheimer's Disease, multiple sclerosis, stroke, trauma, senile dementia, and the like.

In the examples, IGF structural analogs were injected subcutaneously into rats. Ninety minutes later plasma and CSF were withdrawn and analyzed by an ELISA (Table I).

TABLE 1
Selective detection of hIGF-I and its analogs by ELISA

| | OD (450) | |
|---|---|---|
| Sample | Mean ± SEM | P Value |
| Blank | 0.0 ± 0 | |
| Human IGF-I (150 pg) | 0.568 ± .113 | 0.001 |
| Des (1-3) hIGF-I (150 pg) | 0.276 ± .047 | 0.001 |
| Leu 24 hIGF-I (150 pg) | 0.661 ± .072 | 0.001 |
| Human IGF-II (150 pg) | 0.004 ± .017 | 0.959 |
| Insulin (150 pg) | 0.016 ± .006 | 0.903 |
| Rat CSF (extracted) | 0.018 ± .034 | 0.971 |
| Rat Plasma (extracted) | 0.051 ± .037 | 0.818 |

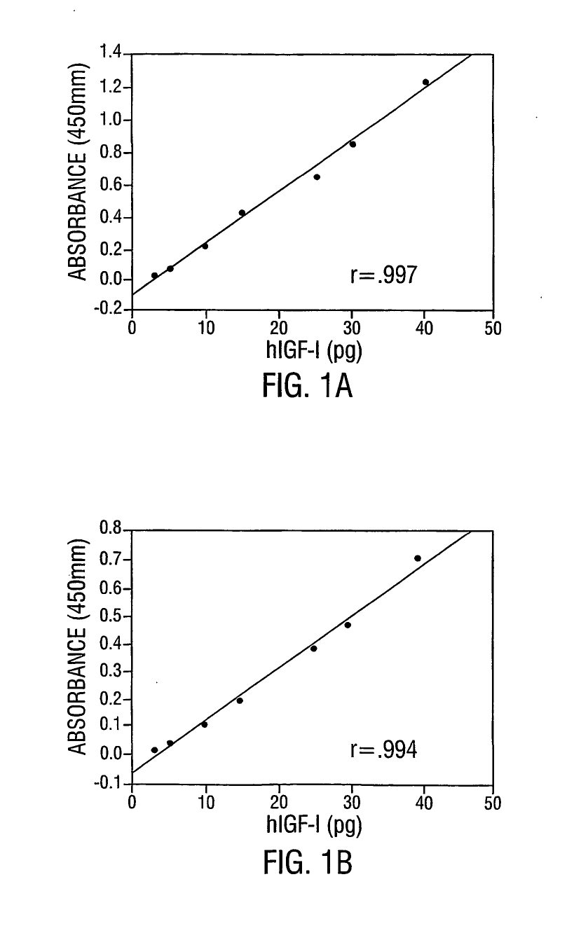

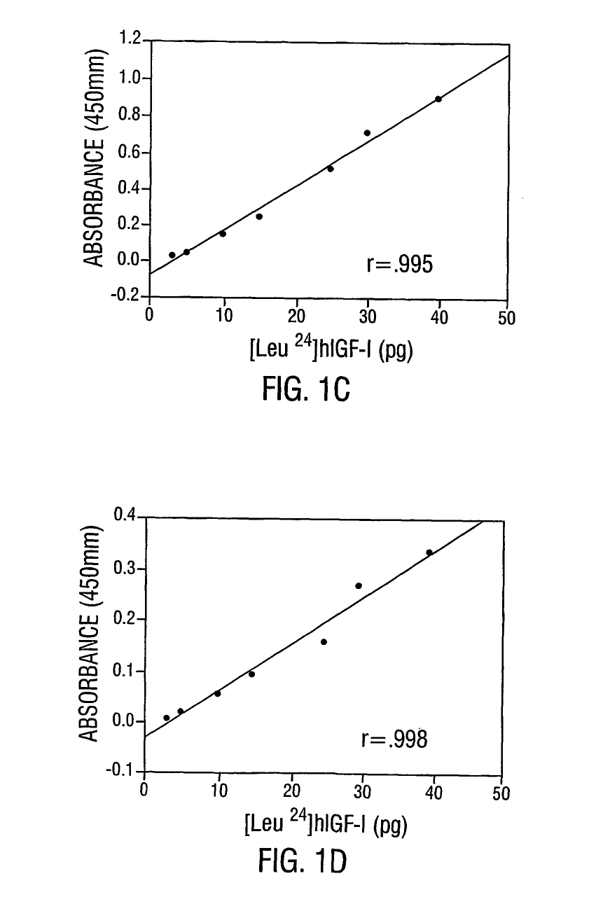

hIGF-I and other proteins were subjected to the ELISA shown in FIG. 1. Untreated rat CSF and plasma were tested at the same volumes assayed throughout these experiments. Note that rat IGF-I, IGF-II, insulin and IGFBP in CSF and plasma do not interfere in the ELISA. Values are means ± SEM of four replicate measurements.

The ELISA detected human IGF-I and IGF structural analogs. However, the ELISA did not detect IGF-II or insulin. Furthermore, nothing in untreated rat CSF or plasma interfered with the ELISA, showing that this test was specific for human IGF-I and IGF structural analogs. In other words, endogenous rat IGF-I, rat IGF-II, rat insulin and other rat substances in CSF or plasma did not interfere in the ELISA. FIG. 1 shows standard ELISA curves for different concentrations of human IGF-I, des(1-3)IGF-I, [Leu24]IGF-I and [Leu60]IGF-I.

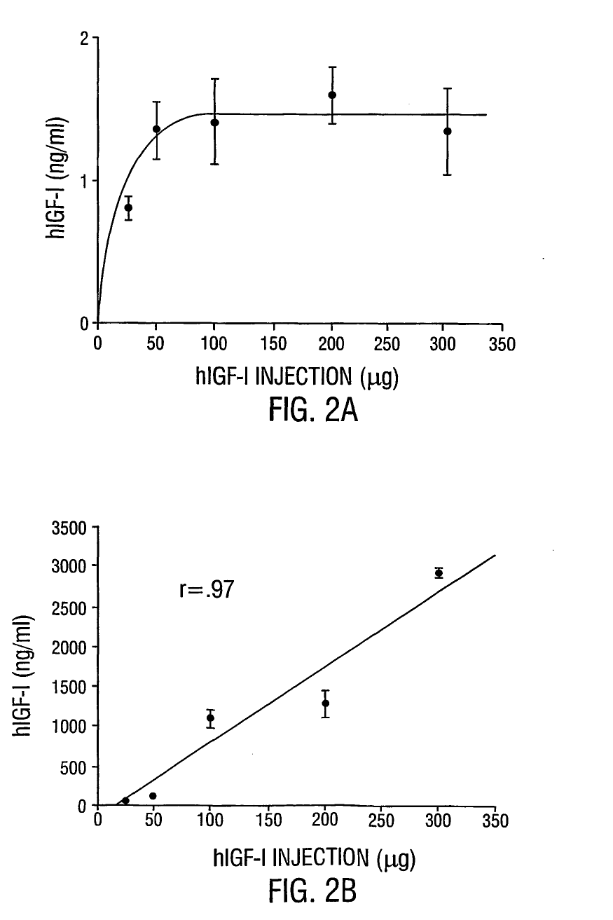

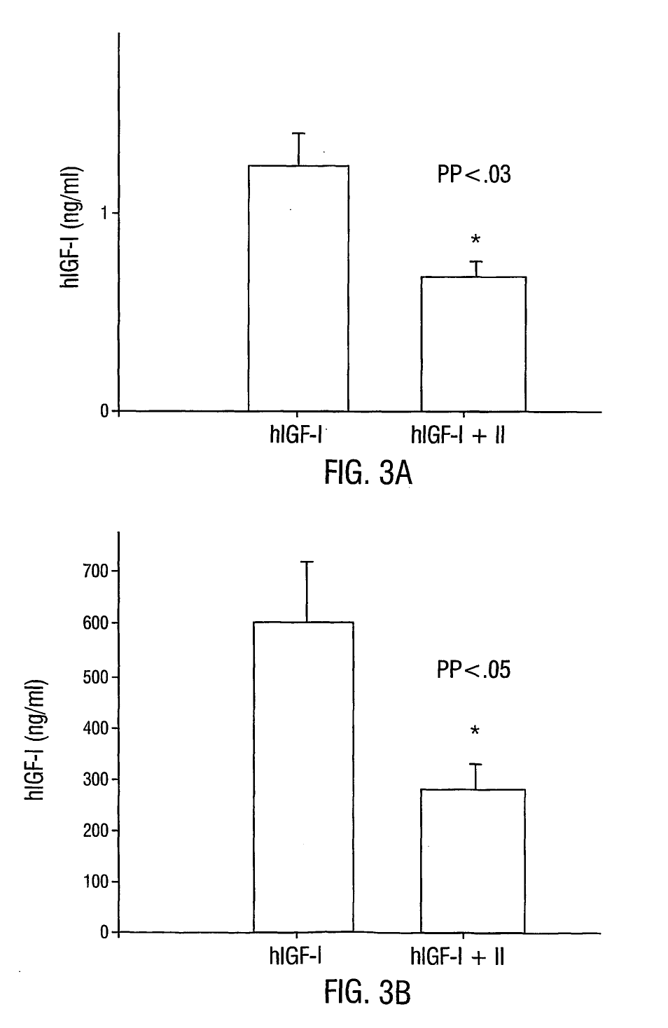

Adult rats were injected subcutaneously with various doses of human IGF-I. FIG. 2 shows that IGF-I in plasma increased linearly with dose. However, IGF-I uptake into CSF saturated with increasing dose, showing that uptake was via an IGF uptake carrier. FIG. 3 shows that IGF-II competed with IGF-I for uptake into CSF.

### EXAMPLES

### Example 1

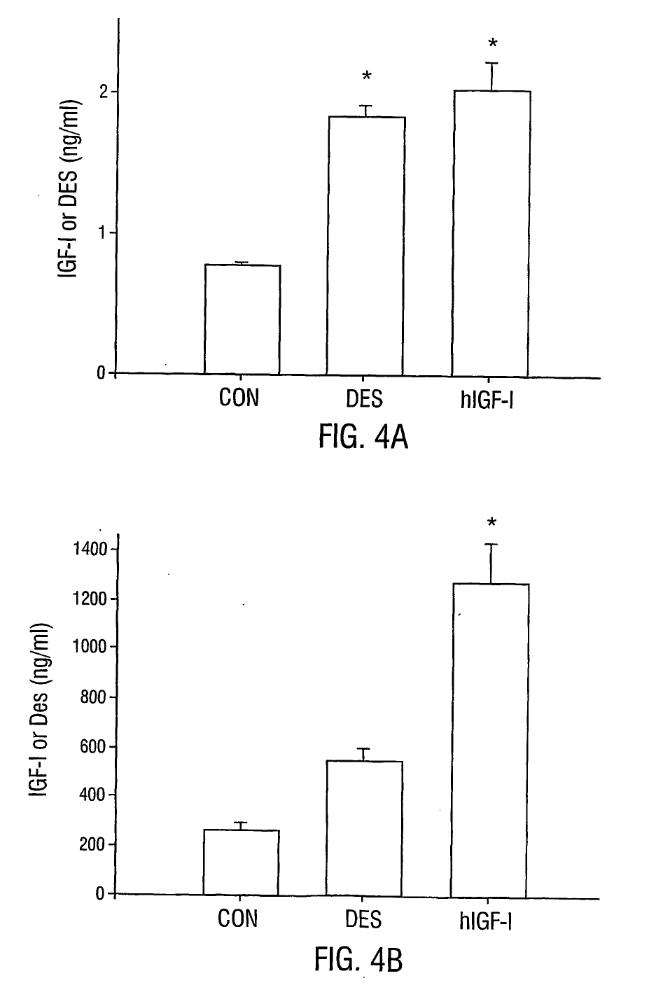

Des(1-3)IGF-I is missing the first 3 amino acids from the N-terminus resulting in at least 25-fold reduced affinity for IGF binding protein-3 (IGFBP-3), IGFBP-4 and IGFBP-5. Binding to IGFBP-1 is reduced as well. Des(1-3)IGF-I binds to the type I IGF receptor, and has enhanced biological activity on neurons. It is more potent due to reduced binding to IGFBP. FIG. 4 shows that des(1-3)IGF-I administered subcutaneously is taken up into cerebrospinal fluid in adult rats. Therefore, binding of IGF and mutant IGFs to IGFBP-1, -3, 4 and -5 is not required for uptake into CSF, and the IGF uptake carrier molecule does not have characteristics of IGFBP-1, -3, -4 or -5.

### Example 2

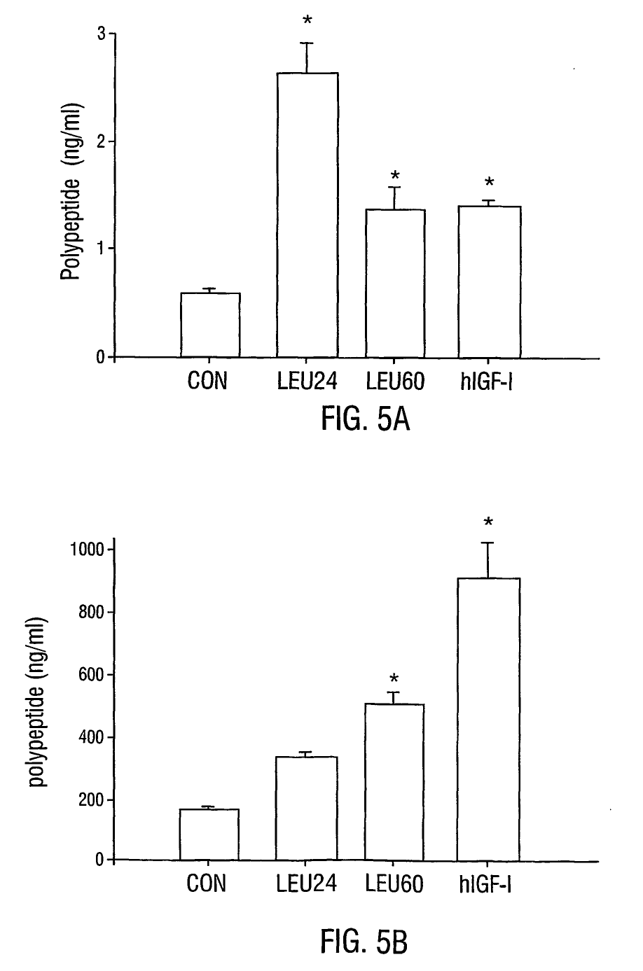

Leu is substituted for Thr at position 24 in [Leu24]IGF-I. Following subcutaneous injection of [Leu24]IGF-I into adult rats, it was readily detected in cerebrospinal fluid (FIG. 5). This, together with Examples 1 and 3, shows that IGF structural analogs with various deletions or substitutions can be taken up into CSF from the circulation.

### Example 3

Leu has been substituted for Tyr at position 60 in [Leu60]IGF-I, which has a 20-fold reduced affinity for the type I IGF receptor. Following subcutaneous injection of [Leu60]IGF-I into adult rats, it was readily detected in cerebrospinal fluid (FIG. 5). This shows that binding to the type I IGF receptor is not necessary for uptake of IGFs, and the IGF carrier molecule does not have characteristics of the type I IGF receptor.

Des(1-3)IGF-I and IGF-I do not bind appreciably to the type II IGF receptor, yet both of these ligands are taken up into CSF following subcutaneous administration. Thus, binding to the type II IGF receptor is not required for uptake of IGFs into CSF, and the IGF carrier molecule does not have characteristics of the type II IGF receptor.

Uptake of insulin-like growth factors (IGFs) from the circulation into cerebrospinal fluid (CSF) is consistent with a transport carrier protein. This carrier protein does not have the same properties as the type I or type II IGF receptors, or IGF binding proteins. Consequently, the carrier has properties unlike that of previously characterized IGF binding molecules.

Therefore, IGF structural analogs are shown to enter the CSF from across the BBB, B-CSF-B and/or B-SC-B in a mammal. This invention has the advantage that mutant IGFs and IGF analogs may be administered from outside of the BBB, B-CSF-B and B-SC-B, and it would not be necessary to use invasive and riskier methods of administration such as intracranial or intrathecal. The risk and cost of surgery and risk of CNS infection may be circumvented by the invention.

1. A method of treating the central nervous system, comprising administering an effective amount of an IGF structural analog to treat or prevent neuronal damage in the central nervous system.

2. The method of claim 1, wherein the neuronal damage to the central nervous system is due to a disorder or disease in the post-birth brain or spinal cord.

3. The method of claim 2, wherein the neuronal damage in the brain is due to Alzheimer's Disease, Parkinson's Disease, AIDS-related dementia, senile dementia, stroke, trauma, cortical-basal ganglionic syndromes, progressive dementia, familial dementia with spastic paraparesis, progressive supranuclear palsy, multiple sclerosis, hepatic encephalopathy, Pick's Disease, Huntington's Disease, diffuse cerebral sclerosis of Schilder, or acute necrotizing hemorrhagic encephalomyelitis.

4. The method of claim 2, wherein the neuronal damage in the brain or spinal cord is a tumor or cancer.

5. The method of claim 1, wherein the IGF structural analog is des(1-3)IGF-I.

6. The method of claim 1, wherein the IGF structural analog is [Arg3]IGF-I, [Leu24]IGF-I, [Leu60]IGF-I, Long R3IGF-I, des(1-6)IGF-II, [Gly1]IGF-II, [Arg6]IGF-II or [Leu27]IGF-II.

7. The method of claim 1, wherein the mutant IGF or IGF analog is administered in an amount from about 0.01 μg/kg/day up to about 4 mg/kg/day.

8. The method of claim 1, wherein the IGF structural analog is administered by nonintracranial and nonintravertebral column administration.

9. A method of treating the central nervous system, comprising nonintracranial and nonintravertebral column administration of an effective amount of an IGF structural analog to treat or prevent neuronal damage in the central nervous system.

10. The method of claim 9, in which the damage is due to a disorder or disease in the central nervous system, except where the disease is amyotrophic lateral sclerosis.

11. The method of claim 9, wherein the damage is due to a tumor or cancer.

12. The method of claims 9, wherein the mutant IGF or IGF analog is des(1-3)IGF-I.

13. The method of claim 9, wherein the IGF structural analog is [Arg3]IGF-I, [Leu24]IGF-I, [Leu60]IGF-I, Long R3IGF-I, des(1-6)IGF-II, [Gly1]IGF-II, [Arg6]IGF-II or [Leu27]IGF-II.

14. The method of claim 9, wherein the mutant IGF or IGF analog is administered in an amount from about 0.01 μg/kg/day up to about 4 mg/kg/day.

15. The method of claim 9, wherein the nonintracranial and nonintravertebral column administration is percutaneous, subcutaneous, intramuscular, intravenous, intraarterial, by inhalation, or intranasal.
